(19) United States
(12) Patent Application Publication
(10) Pub. No.: US 2022/0323104 A1
(43) Pub. Date: Oct. 13, 2022

(54) INTRA-BODY PASSAGEWAY DEVICE AND METHOD

(71) Applicant: Yaron RAITER, Amsterdam (NL)

(72) Inventor: Yaron RAITER, Amsterdam (NL)

(21) Appl. No.: 17/639,604

(22) PCT Filed: Sep. 3, 2020
(86) PCT No.: PCT/IL2020/050965

§ 371 Date: Mar. 2, 2022

Related U.S. Application Data
(60) Provisional application No. 62/894,983, filed on Sep. 3, 2019.

(51) Int. Cl. A61B 17/34

(52) U.S. Cl. CPC A61B 17/3423

(57) ABSTRACT

An intra-body system and method of inserting a flexible sleeve as a passageway into an internal organ. The method comprises inserting a channel into an organ by perforating the wall of the organ; releasing a distal fixation portion of the passageway sleeve inside the organ; fixating the distal fixation portion to the wall of the organ; pulling a proximal portion of the passageway sleeve outside the body to stretch the sleeve. The stretched sleeve: a) generates a passageway between the proximal end of the sleeve at the outer body portion and the distal portion of the sleeve inside the organ, and b) seals the perforation rim at the organ.

### RELATED APPLICATION/S

This application claims the benefit of priority under 35 USC §119(e) of U.S. Provisional Patent Application No. 62/894,983 filed 3 Sep. 2019, the contents of which are incorporated herein by reference in their entirety.

### FIELD AND BACKGROUND

The present invention, in some embodiments thereof, relates to an intra-body passageway device and, more particularly, but not exclusively, to a device and method for generating passage into an organ located in the abdomen.

### SUMMARY

The following describes some examples of embodiments of the invention. Some example of the invention are described herein and an embodiment may include features from more than one example and/or fewer than all features of an example.

According to an aspect of some embodiments of the present invention there is provided a method of inserting a passageway sleeve into an internal organ. The method includes: inserting a channel into an organ by perforating the skin and the wall of the organ; releasing the passageway sleeve inside the organ; fixating a fixation portion of the passageway sleeve to the wall of the organ; removing the channel outside the body; and expanding the passageway sleeve to produce a passageway between outside the body and inside the organ. According to some embodiments, the method includes advancing a tool into the organ from a proximal portion of the passageway sleeve.

According to an aspect of some embodiments of the present invention there is provided a method of inserting a flexible sleeve as a passageway into an internal organ. The method comprise: inserting a channel into an organ by perforating the wall of the organ; releasing a distal fixation portion of the passageway sleeve inside the organ; fixating the distal fixation portion to the wall of the organ; pulling a proximal portion of the passageway sleeve outside the body to stretch the sleeve, wherein the stretched passageway sleeve generates a passageway between the proximal end of the sleeve at the outer body portion and the distal portion of the sleeve inside the organ. According to some embodiments, the stretched passageway sleeve seals the perforation rim at the organ.

According to some embodiments of the invention, the method comprises advancing the fixation portion inside the channel to a release port formed at a wall of the channel, and releasing the fixation portion out of the release port.

According to some embodiments of the invention, the method comprises securing the proximal portion of the sleeve to the body.

According to some embodiments of the invention, the method comprises removing content out of the internal organ via the passageway.

According to some embodiments of the invention, the method comprises removing the channel outside the organ and the body prior to the pulling of the proximal portion.

According to some embodiments of the invention, the method comprises pre-fixating the organ to the body at one or more locations, prior to inserting the channel. According to some embodiments of the invention, the method comprises reducing the size of the fixation portion of the passageway sleeve, and removing the passageway sleeve outside the organ.

According to an aspect of some embodiments of the present invention there is provided an intra-body passageway system, the system comprises a channel and a passageway sleeve. The channel comprises an elongated barrel having a sleeve release port, and a tip at a distal end of the channel, sized and shaped to penetrate an organ. The passageway sleeve is inserted at least partially within the barrel and comprises a flexible wall, between a distal end and a proximal end of the sleeve, and a resilient fixation portion, integrated to the distal end of the sleeve. According to some embodiments, the fixation portion is expandable out of the channel, via the sleeve release port.

According to some embodiments of the invention, the portion of the barrel configured to penetrate the organ has a maximal width of at least 10 mm.

According to some embodiments of the invention, the channel comprises a plunger, and the sleeve is slideable by the plunger.

According to some embodiments of the invention, the wall of the sleeve has a conical shape between the fixation portion and the proximal portion of the sleeve, when unfolded.

According to some embodiments of the invention, the system comprises a sleeve fastener shaped to couple to a proximal portion of the sleeve to secure the sleeve to the body. In some embodiments of the invention, the sleeve comprises a tread, and the sleeve fastener comprises a matching tread to screw the sleeve fastener on the tread of the sleeve.

According to some embodiments of the invention, the barrel comprises a sleeve orientation flange, protruding from the sleeve release port to control the orientation of the fixation portion, by interrupting the expansion of the fixation portion out of the sleeve release port.

According to some embodiments of the invention, the tip is pivotally coupled to the barrel. According to some embodiments of the invention, the tip is detachable of the barrel.

According to some embodiments of the invention, the system comprises a balloon coupled to the distal portion of the sleeve. In some embodiments, the balloon comprises anchors, shaped to secure the balloon to the wall of the organ.

According to an aspect of some embodiments of the present invention there is provided a method of inserting a passageway sleeve into an internal organ. The method includes: diagnosing abdominal symptoms in a patient; inserting a channel into an organ by perforating the skin and the wall of the organ without anesthesia; securing a passageway sleeve inside the organ; removing the channel outside the body; and accessing the organ by an operator or a tool via the passageway sleeve.

According to some embodiments of the invention, the method includes removing the passageway from organ.

According to some embodiments of the invention, the method includes creating a fistula by the passageway.

According to some embodiments of the invention, the time between the diagnosing, and the accessing is less than 1 hour.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed than the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified flowchart of a medical procedure, according to some embodiments of the invention;

FIG. 2A-2B are simplified flowcharts of fixating a passageway into an abdomen organ, according to some embodiments of the invention;

FIGS. 3A to 3E are simplified side view simplified illustrations of a system, according to some embodiments of the invention;

FIG. 4 is a simplified side view schematic illustration of a passageway insertion system, according to some embodiments of the invention;

FIGS. 5A and 5B are schematic simplified side views of an intra-body passageway insertion system, according to some embodiments of the invention;

FIGS. 6A to 6B are simplified illustrations of a side view of a sleeve, according to some embodiments of the invention;

FIG. 6C is a simplified illustration of a side view of a sleeve fastener, according to some embodiments of the invention;

FIG. 6D is a simplified illustration of a side view of a sleeve fastener superimposed on a passageway sleeve, according to some embodiments of the invention;

FIG. 7A is a simplified illustration of a side view of a sleeve, according to some embodiments of the invention;

FIG. 7B is a simplified illustration of a side view of a sleeve fastener, according to some embodiments of the invention;

FIG. 7C is a simplified illustration of a side view of a sleeve fastener superimposed on a passageway sleeve, according to some embodiments of the invention; and

FIGS. 8A to 8D are simplified side view illustrations of an organ pre-fixation system, according to some embodiments of the invention.

### DESCRIPTION OF SPECIFIC EMBODIMENTS OF THE INVENTION

The present invention, in some embodiments thereof, relates to an intra-body passageway device and, more particularly, but not exclusively, to a device and method for generating passageway into an organ located in the abdomen.

### Overview of the Invention

An aspect of some embodiments of the invention relates to a secured flexible passageway sleeve placed into an organ by a channel inserted into the organ via an incision at the abdomen.

According to some embodiments, the passageway is inserted and secured to the organ by a non-laparoscopic procedure. In some embodiments, the passageway is inserted and secured to the organ by a procedure, which is not an open surgery. In some embodiments, the procedure does not require an operational room and is performed for example in an emergency trauma room.

According to some embodiments, the passageway spans between a distal end of the sleeve, which is secured to an inside surface of the wall of the organ, and a proximal end of the sleeve secured to an outer body portion.

According to some embodiments, one end of the sleeve has a distal expandable fixation element to expand and secure a portion of the sleeve inside the organ. In some embodiments, the expandable element is a resilient ring coupled to a distal end of the sleeve placed inside the channel and self-expands out of the channel.

According to some embodiments, securing the sleeve outside the body is by stretching a proximal portion of the sleeve outside the body past the channel perforation area. In some embodiments, securing of the sleeve includes fixating the proximal portion of the sleeve to the skin outside the body.

According to some embodiments, the sleeve seals the organ at the perforation rim created by the insertion of the channel into the organ. In some embodiments, sealing is by applying an outward radial force on the surface of the organ at the perforation rim. In some embodiments, sealing is by applying an axial force on the surface of the organ at the perforation rim. In some embodiments, sealing is by applying a combination of a radial force and an axial force on the wall of the organ at the perforation rim from the inner side of the organ. In some embodiments, at least a portion of the sleeve is axially elastic. In some embodiments, at least a portion of the sleeve is radially pliable.

According to some embodiments, the inside portion of the sleeve is inserted into the organ through the channel. In some embodiments, the channel has a release port for releasing the fixation portion of the sleeve out of the channel after inserted into the organ. In some embodiments, the release port is located at a side wall of the channel. In some embodiments, the release port is located at a distal end of the channel. In some embodiments, the release port is located at both the distal and the wall of the channel. In some embodiments, the channel is a trocar.

According to some embodiments, the channel accommodates at least a portion of the sleeve prior to insertion of the channel into the body. In some embodiments, the channel accommodates the sleeve between inserting the channel into the organ and the releasing of the fixation portion of the sleeve inside the organ. In some embodiments, a sleeve magazine is used to place the sleeve inside the channel. In some embodiments, the sleeve magazine is shaped to fix the fixation portion in the channel. In some embodiments, a sleeve is placed within the channel as part of a prepared to use kit.

According to some embodiments, a proximal portion of the sleeve is disposed partially or completely outside the channel while securing a distal portion inside the organ. In some embodiments, the sleeve is inserted into the channel during the procedure. In some embodiments, the proximal portion is secured outside the body, prior to securing the distal potion inside the organ.

According to some embodiments, the channel includes an indicator detectable by a detection device for guiding the channel inside the body by an operator. In some embodiments, the indicator is metallic. In some embodiments, the indicator is coupled to the tip of the channel. In some embodiments, the indicator has florescent characteristics.

An aspect of some embodiments of the invention relates to placing a secured flexible passageway sleeve into an organ by a tubular channel wider than a needle.

According to some embodiments, the channel is released outside the abdomen via the same incision.

According to some embodiments, the maximal width of the channel is less than the maximal diameter of the opening of a secured passageway. In some embodiments, the maximal width of the channel is less than the maximal diameter of the opening of the fixation portion of secured passageway. In some embodiments, the maximal width of the perforation for inserting the channel into the organ is at least 5 to 10 mm less than the maximal diameter of the opening of the fixation portion of secured passageway. In some embodiments, the maximal width of the portions of the channel inserted inside the body is between 5 and 20 mm smaller than the maximal diameter of a secured passageway. In some embodiments, the maximal width of the portions of the channel inserted inside the body is between 10 and 15 mm smaller than the maximal diameter of a secured passageway. In some embodiments, the maximal width of the portions of the channel inserted inside the body is between 5 and 20 mm smaller than the maximal diameter of a secured passageway at its distal end. In some embodiments, the maximal width of the portions of the channel inserted inside the body is between 10 and 35 mm smaller than the maximal diameter of a secured passageway at its proximal end. According to some embodiments, the channel has a maximal diameter of at least 10 mm. In some embodiments, the channel has a maximal diameter of at least 5 mm. In some embodiments, the channel has a maximal diameter of at least 15 mm. In some embodiments, the diameter of the fixation portion of the secured passageway is at least 10 mm. In some embodiments, the diameter of the fixation portion of the secured passageway is at least 4 mm. In some embodiments, the diameter of the fixation portion of the secured passageway is at least 15 mm.

An aspect of some embodiments of the invention relates to inserting a passageway sleeve into an internal organ without anesthesia. In some embodiments, the organ is in the abdomen.

According to some embodiments, inserting is performed after diagnosing abdominal symptoms. In some embodiments, inserting is performed in less than 1 hours after the diagnosing. In some embodiments, inserting is performed in less than 2 hours after the diagnosing. In some embodiments, inserting is without making an incision.

According to some embodiments, inserting is followed by securing a passageway sleeve inside the organ and removing the channel outside the body. In some embodiments, the organ is treated by accessing the organ by an operator or a tool via the passageway. In some embodiments, accessing the organ via the passageway is performed in less than 2 hours after the diagnosing.

A potential advantage of a system and method of the above aspects of the invention is providing an access to the bowel under radiological/imaging/virtual reality guidance with or without additional means/procedures such as open-surgery, laparoscopic, and endoscopic. A potential usage of a system and method of the above aspects of the invention is generating an opening into the bowel to treat/diagnose bowel related conditions and/or stabilize an acute/emergency medical condition.

Before explaining at least one embodiment of the invention in detail, it is to be understood than the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Ecosystem

Bowel/intestinal obstruction occurs when the small or large bowel is blocked due to mechanical or functional reasons such as adhesions, hernias, volvulus, endometriosis, inflammatory bowel disease, appendicitis, tumors, diverticulitis, intussusception, and others. The blockage can be partial or total, it prevents passage of digested food and fluids. The food, fluids, gastric acids, and/or gas build up behind the site of the blockage. If enough pressure builds up the bowel can rupture, leaking harmful bowel contents and bacteria into the abdomen cavity. This is a life-threatening complication. Intestinal obstruction is knows in elderly, who tend to sit for long periods with reduced body movements.

Emergency bowel obstruction surgery often requires pre- and post-surgery hospitalization. Prolonged pre-surgery hospitalization might occur due to lack of operation room/staff and/or patient severe medical condition which delays the surgery, thus the patient safety/wellbeing is at risk, life-threatening is a known complication. The surgery is done in the operation room while the patient is under general anesthesia, the surgeon makes a cut in the belly to see the bowels. This method is regard as laparotomy.

In some cases, the surgery is done using a laparoscopy method in the operation room while the patient is under general anesthesia and smaller cuts are used to insert the operational tolls without a cut in the belly. The surgeon locates the blocked area and try to unblock it, any damaged parts of the bowel will be repaired or removed. In case a section is removed the healthy ends will be reconnected. Sometimes when part of the bowel is removed, the ends cannot be reconnected and if this happens the surgeon will bring one end out through an opening in the abdomen wall and generate a bowel opening on the abdomen wall, this is called stoma. Pre- and post-surgical complications include among others; wound infection, anastomotic leak, stoma complications, perforation, intra-abdomen abscess, sepsis, repeat surgery, and death.

An invasive treatment may require the stabilization of the patient, which may take a long period and a delay in a treatment when time can be critical.

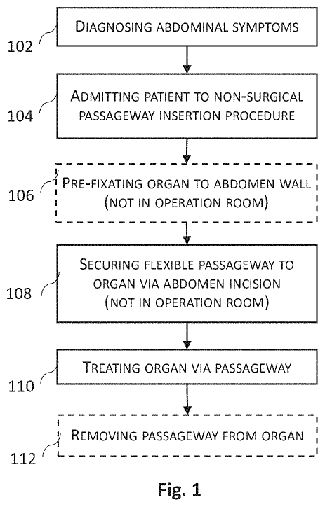

Turning to FIG. 1, which is a simplified chart of a medical procedure, according to some embodiments of the invention.

According to some embodiments, the organ treatment procedure described hereinafter does not require an open-surgery. In some cases, this implies than the organ treatment procedure is of a non-surgical category. In some embodiments, the procedure does not require an operational room and is performed, for example, in an emergency trauma room, which may reduce standard requirements that exist in open-surgery. In some cases, this may reduce standard requirements and treatment protocols that exist in the current operational clinical practice setting. In some cases, this may reduce time to treatment, e.g. treatment in 1-2 hours after an initial diagnosis. In some embodiments, procedure is done in an operational room.

As shown in the example embodiment of FIG. 1, the organ treatment procedure includes some diagnostic and procedural steps.

Some example of steps that precede the organ treatment procedure can be:

Diagnosing 102 abdomen symptoms. According to some embodiments, diagnosing 102 is non-surgical. If required, diagnosing 102 is followed by admitting 104 patient to a non-surgical abdomen insertion passageway procedure. Since according to some embodiments the abdomen passageway insertion procedure can be facilitated in a non-surgery room, for example out-side the operational room, admitting 104 can be to a facility not-having operational rooms or not-having surgery personnel.

According to some embodiments, for example in case the organ is located deep or far of the wall of the abdomen, the passageway insertion procedure includes pre-fixating 106 the organ to abdomen wall. In some embodiments, pre-fixating 106 is repeated to secure the organ in one or more locations.

According to some embodiments, the passageway insertion procedure includes securing 108 a flexible passageway to organ via an abdomen incision. In some embodiments, incision location is determined according to pre-fixating 106. In some embodiments, pre-fixating 106 is optional and securing 108 does not require fixation of the organ to the abdomen wall. In some embodiments, incision location is based on prior imaging of the abdomen. In some embodiments, passageway insertion location is based on diagnosing 102.

According to some embodiments, treating 110 the organ in the abdomen via the passageway is done in a medical facility. In some embodiments, treating 110 is out-side a medical facility, such as home and home care. In some embodiments, treating 110 is done 1-2 hours after diagnosing 102. In some embodiments, treating 110 is done in less than an hour after diagnosing 102.

According to some embodiments, the organ treatment procedure includes removing 112 passageway from organ. In some embodiments, removing 112 is performed without an open-surgery. In some embodiments, removing is performed immediately after treating 110. In some embodiments, removing 112 is performed in another session hours, days, weeks, or months after treating 110 the organ.

### Fixating Passageway Procedure

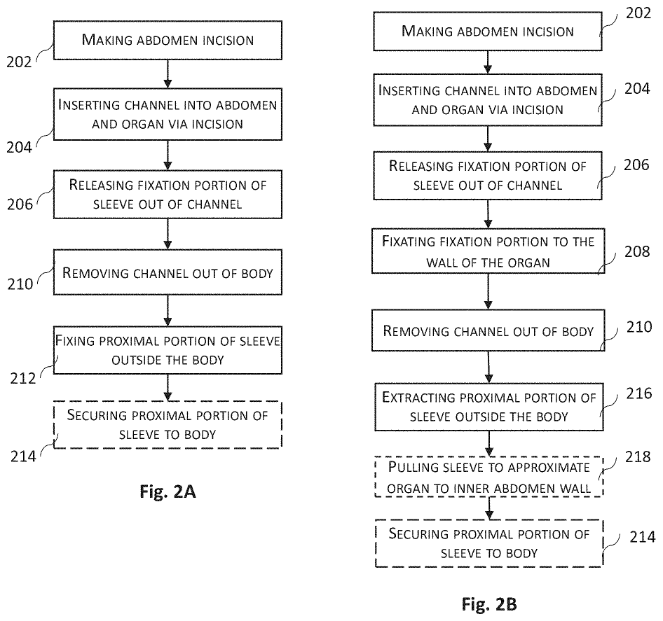

FIGS. 2A-2B show example embodiments, of steps in a procedure of fixating a passageway into an abdomen organ, according to some embodiments of the invention.

According to some embodiments, the procedure of fixating a passageway into an abdomen organ is done via an abdomen incision and not by an open-surgery. In some examples, the procedure include the following steps shown in FIG. 2A:

Making 202 an abdomen incision. In some embodiments, the location of the incision is selected to be between two or more locations in which the organ is pre-fixated to the abdomen wall (e.g. by a preceding organ pre-fixation procedure described elsewhere herein).

Inserting 204 a guiding channel into abdomen and into an organ via incision, to guide a passageway sleeve from outside the body into the organ. In some embodiments, the guiding channel is in the form of a trocar. In some embodiments, inserting 204 is through an incision without securing the organ to abdomen wall.

Releasing 206 a fixation portion of the sleeve out of channel.

According to some embodiments, releasing 206 follows a sliding the passageway sleeve inside the channel. In some embodiments, releasing 206 follows sliding a distal portion of the flexible passageway sleeve inside the channel. In some embodiments, sliding is by pushing a plunger inside the channel.

Removing 210 the guiding channel out of the body. According to some embodiments, removing 210 is performed while the fixation portion of the sleeve is disposed within the organ.

Fixating 212 the proximal portion of the sleeve outside the body.

According to some embodiments, fixating 212 of the proximal portion of the sleeve is performed together with removing 210 of the channel. In some embodiments, the proximal portion of the sleeve is connected to the channel, so that fixating 212 and removing 210 are synchronized. In some embodiments, fixing 212 includes stretching the wall of the sleeve to maintain pressure between the fixation portion located inside the organ, and the proximal portion of the sleeve on the abdomen skin.

Optionally, fixating 212 is followed by securing 214 the proximal portion of the sleeve to the body. In some embodiments, securing 214 is to a base located on the abdomen skin. In some embodiments, securing 214 is by coupling a sleeve fastener to the proximal portion of the sleeve.

According to some embodiments, the procedure includes intra-body imaging through the channel prior to releasing 206, for example to identify target location, or to assess and diagnose the area/organ of interest. In some embodiments, intra-body imaging is performed after releasing 206, for example to check for proper fixation of the sleeve inside the organ. In some embodiments, the procedure include imaging from outside the body, for example to identify an insertion site location. In some embodiments, imaging using one or more imaging devices such as ultrasound, laparoscope, and endoscope, is used during inserting 204 of the channel into the organ, and once located inside the target organ, the imaging tool is removed.

Another example of steps performed during the procedure is shown in FIG. 2B. The example of FIG. 2B differs of the procedure shown in FIG. 2A by having the following steps:

208 Fixating the fixation portion to a wall of the organ. In some embodiments, fixating 208 include inflating an expandable portion of the sleeve disposed inside the organ. In some embodiments, fixating 208 include inflating a balloon inserted into the organ by the channel. In some embodiments, fixating 208 potentially prevents leakage from the organ into the abdomen or outside the body.

216 extracting the proximal portion of the sleeve outside the body. This step is required, for example when a proximal portion of the sleeve is located inside the channel.

According to some embodiments, extracting 216 is performed together with removing 210. In some embodiments, the proximal portion of the sleeve is connected to the channel, so that extracting 216 and removing 210 are synchronous.

218 Pulling the sleeve to approximate the organ to the inner abdominal wall. According to some embodiment, pulling 218 of the sleeve pulls the fixation portion and thereby the organ it is attached to. In some embodiments, pulling 218 urges the organ against the inner abdominal wall. In some embodiments, pulling 218 includes stretching the wall of the sleeve to apply a radial force at internal tissues, for example to seal the passageway insertion location into the organ. In some embodiments, stretching the wall of the sleeve defines an open passageway.

214 Optionally securing proximal end of sleeve to body. According to some embodiments, securing 214 maintains the organ in a fixed position in respect to the skin on the body. In some embodiments, securing 214 potentially stabilizes the proximal end of the passageway on the body.

According to some embodiments, fixating 208 of the fixation portion follows any one of the following steps: removing 210, extracting 216, pulling 218, and securing 214, so than the proximal portion of the sleeve is handled prior to completing the fixation of the distal portion.

According to some embodiments, the sleeve is removed via surgery. In some embodiments, removing the sleeve is by removing the fixation portion of the sleeve. In some embodiments, removing of the sleeve is by a procedure using a device that decrease the size of the fixation portion from the outside of the organ without a surgery. In some embodiments, removing the sleeve outside the organ and body, is followed by a procedure to close the opening for example by suturing the opening.

Fixating Passageway System

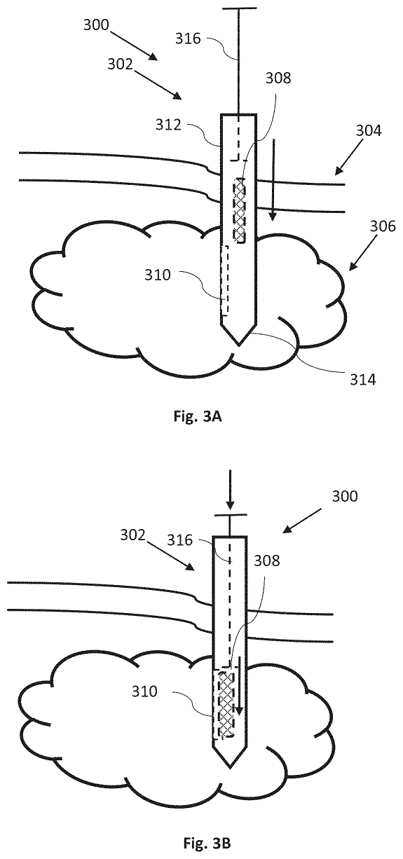

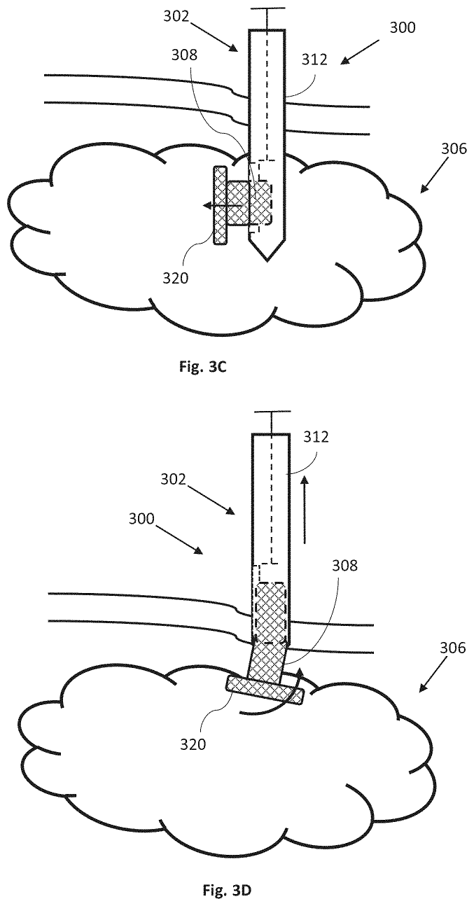

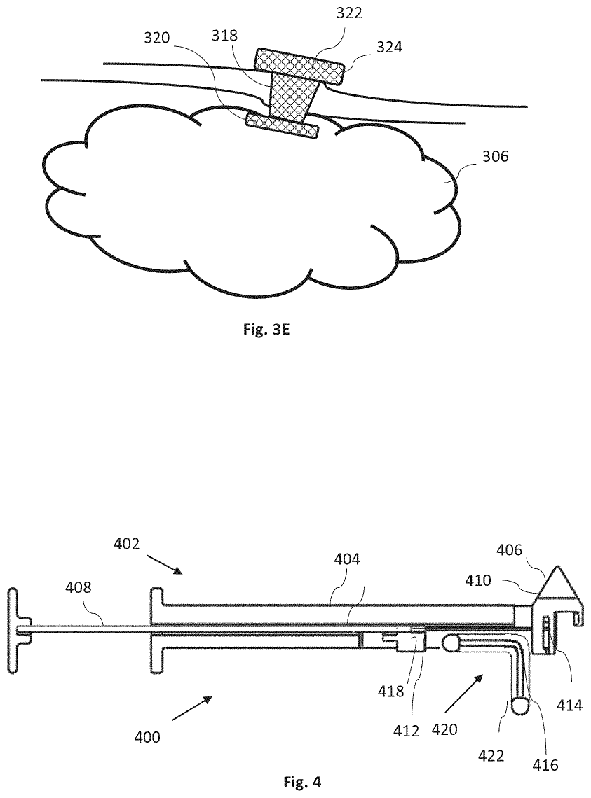

Turning to FIGS. 3A-3E show side view simplified illustrations of a system used in a procedure of fixating a passageway into an abdomen organ, according to some embodiments of the invention.

The example embodiments of FIGS. 3A-3E show a system 300 that includes a channel 302 configured to be inserted through the skin 304 into an organ 306 in the abdomen.

FIGS. 3A-3B show an example embodiment of inserting a passageway sleeve 308 by channel 302 into organ 306. FIGS. 3C-3D show an example embodiment of releasing sleeve 308 into organ 306 through a sleeve release port 310, after disposing at least a portion of sleeve 308 insider organ 306.

According to some embodiments, sleeve 308 is encompassed within channel 302 prior to releasing sleeve 308 into organ 306. In some embodiments, for example as shown in FIG. 3B, sleeve 308 is pushed distally inside channel 302, towards sleeve release port 310. In some embodiments, channel 302 includes a plunger 316 slideable within channel 302 for pushing sleeve distally. In some embodiments, prior to releasing sleeve 308 into organ 306, sleeve 308 is coupled to the wall of channel 302. In some embodiments, sleeve 308 is coupled to an outside wall of channel 302 prior to releasing sleeve 308 into organ 306.

FIG. 3E, shows an example of a passageway 318 formed between outside the body into organ 306, after releasing channel 302 (shown in FIG. 3D). Passageway 318 is formed by having a distal portion 320 of sleeve 308 disposed inside organ 306 and a proximal portion 322 of sleeve 308 disposed outside the body. In some embodiments passageway 318 is a bi-directional passageway to allow inserting/extracting of materials into/outside the organ. In some embodiments, passageway 318 is configured for inserting tools into the organ. In some embodiments, passageway 318 includes a one-way valve directed to allow inserting material into the organ. In some embodiments, passageway 318 includes a one-way valve directed to allow releasing material out of the organ.

According to some embodiments, the minimal diameter of passageway 318 at distal portion 320 is between 2 and 30 mm. In some embodiments, the minimal diameter of passageway 318 at distal portion 320 is between 5 and 20 mm. In some embodiments, the minimal diameter of passageway 318 at distal portion 320 is between 10 and 15 mm.

According to some embodiments, the minimal diameter of passageway 318 at proximal portion 322 is between 2 and 35 mm. In some embodiments, the minimal diameter of passageway 318 at proximal portion 322 is between 5 and 20 mm. In some embodiments, the minimal diameter of passageway 318 at proximal portion 322 is between 10 and 15 mm. In some embodiments, the minimal diameter of passageway 318 at proximal portion 322 is larger than the minimal diameter of passageway 318 at distal portion 320 by 0 to 5 mm. In some embodiments, the minimal diameter of passageway 318 at proximal portion 322 is larger than the minimal diameter of passageway 318 at distal portion 320 by 5 to 10 mm. In some embodiments, the minimal diameter of passageway 318 at proximal portion 322 is larger than the minimal diameter of passageway 318 at distal portion 320 by 10 to 15 mm.

### Channel/Trocar

As schematically shown in FIGS. 3A-3D, channel 302 includes a longitudinal barrel 312 used to guide a passageway sleeve 308 from outside the body into the organ 306.

According to some embodiments, channel 302 is at least partially rigid and can perforate the wall of organ 306. According to some embodiments, channel 302 includes a rigid distal tip 314 shaped to penetrate organ 306. In some embodiments, tip 314 is tapered. In some embodiments, tip 314 is conical. According to some embodiments, channel 302 is a trocar.

According to some embodiments, barrel 312 has a proximal opening for inserting sleeve 308 and accommodating sleeve 308 inside barrel 312. In some embodiments, body 316 accommodates sleeve 308 prior to inserting channel 302 into the body.

According to some embodiments, as shown for example in FIGS. 3C-3D, at least a portion of sleeve 308 is released out of barrel 312 after inserted into organ 306. In some embodiments, barrel 312 has a sleeve release port 310 to release a portion of sleeve 308 out of barrel 312. In some embodiments, sleeve is released out of release port 310 in a direction which is transverse to the axial longitudinal direction of barrel 312. In some embodiments, release port 310 is located at a side wall of barrel 312. In some embodiments, barrel 312 has a sustained release mechanism, to release at least a portion of sleeve 308 outside the barrel 312.

According to some embodiments, barrel 312 is long enough to have the proximal opening of barrel 312 exposed outside the body, while having a portion of body 316 inserted into organ 306. In some embodiment, barrel 312 is long enough to have the proximal opening of barrel 312 exposed outside the body, while having release port 310 inserted into organ 306. In some embodiments, the length of barrel 312 is between about 4 and 20 cm. In some embodiments, the length of barrel 312 is between about 6 and 15 cm. In some embodiments, the length of barrel 312 is between about 8 and 12 cm.

In some embodiments, channel 302 has a lumen used to insert an imaging tool therethrough into the body. In some embodiments, sleeve 308 is inserted to channel 302 after removing the imaging tool. In some embodiments, the imaging tool, such as endoscope, can be attached to the outer side of the channel 302.

Turning to FIG. 4, which shows a schematic side view of an example of an intra-body passageway system, according to some embodiments of the invention.

Intra-body passageway system 400, has a channel 402, which is structurally similar to channel 302 described elsewhere herein. For example, channel 402 has a barrel 404, a tip 406, and a plunger 408.

According to some embodiments, tip 406 is coupled to barrel 404 at the distal end of barrel 404. In some embodiments, tip 406 is pivotally coupled to barrel 404. In some embodiments, tip 406 is detachable of barrel 404. In some embodiments, tip 406 is detachable by rotating tip 406 in respect to barrel 404. In some embodiments, tip 406 is detachable by applying a force on tip 406, for example by pulling tip 406 against the wall of an organ. A potential advantage of having tip 406 detachable or rotatable is reducing the risk of damaging the wall of the organ after tip 406 is inside the organ.

In some embodiments, the outside surface of tip 406 is free of protruding elements of the conical surface, to reduce damage to tissues when rotating channel 402 while inserted into the body.

According to some embodiments, channel 402 includes an indicator 410 detectable by a detection device for guiding the channel inside the body by an operator. In some embodiments, indicator 410 is metallic and is detectable by a detection device sensitive to metals and/or magnetic field. In some embodiments, indicator 410 is coupled to tip 406.

As shown in FIG. 4, channel 402 includes a release port 412 formed as a side wall of barrel 404. In some embodiments, port 412 is an elongated opening formed at the wall of barrel 404.

According to some embodiments, system 400 includes a sleeve 420 coupled to side wall of barrel 404 within release port 412. In some embodiments, sleeve 420 is coupled to side wall of barrel 404 within release port 412 prior to inserting channel 402 into the body. In some embodiments, sleeve 420 is coupled to side wall of barrel 404 within release port 412 prior to inserting channel 402 into the organ. In some embodiments, sleeve 420 is coupled to side wall of barrel 404 within release port 412 by moving sleeve 420 along channel 402 using plunger 408 after inserting channel 402 into the organ.

According to some embodiments, movable tip 406 is controlling the release of sleeve 420 out of channel 402. According to some embodiments, tip 406 includes a chamber 414 formed at a proximal side of tip 406. In some embodiments, securing sleeve 420 in port 412 is by holding a portion of sleeve 420 within chamber 414. In some embodiments, releasing sleeve 420 out of port 412, is by moving tip 406 and herby releasing sleeve 420 out of chamber 414. In some embodiments, tip 406 includes a side wall 416, shaped and sized to push a sleeve portion 422 away of channel 402 to release sleeve 420 outside channel 402.

According to some embodiments, tip 406 is detachable. In some embodiments, detaching tip 406 reduces damage to the tissues of the organ, for example during a removal of channel 402 outside the organ.

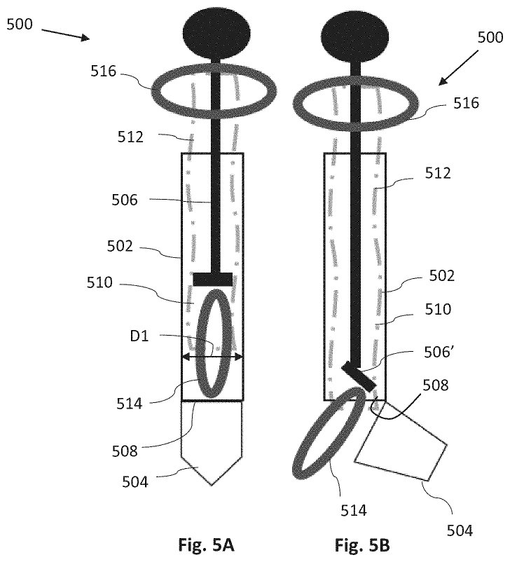

Turning to FIGS. 5A and 5B, which show schematic side views of an example of an intra-body passageway system, according to some embodiments of the invention.

Intra-body passageway system 500 includes a channel 502, which is structurally similar to channel 302 described elsewhere herein. For example, channel 502 has a tip 504 and a plunger 506.

One of the differences between the system 300 and system 500 is having a release port 508 located at a distal end of channel 502. As shown in FIG. 5B, system 500 releases distal portion 514 of sleeve 510 outside channel 502 in an axial direction.

According to some embodiments, plunger 506 has a slanted distal surface 506′ to provide distal portion 514 of sleeve 510 a sideway movement outside channel 502. In some embodiments, distal surface 506′ is movable to change its angular orientation.

In some embodiments, tip 504 has one or more walls that unfold to generate a release port 508 at tip 504.

In some embodiments, tip 504 is pivotally coupled to channel 502. In some embodiments, tip 504 is detachable of channel 502. In some embodiments, tip 504 is detachable by rotating tip 504. In some embodiments, tip 504 is detachable by applying a force on tip 504. A potential advantage of having tip 504 detachable or rotatable is reducing the risk of damaging the wall of the organ after tip 504 is inside the organ.

According to some embodiments, sleeve 510 has a proximal portion 516 located outside channel 502, while inserting sleeve 510 into the organ. Distal portion 514 is movable axially within channel 502 by plunger 506.

According to some embodiments, the portions of the channel inserted into the body have a maximal diameter D1 of at least 10 mm. In some embodiments, the portions of the channel inserted into the body have channel has a maximal diameter D1 of at least 5 mm. In some embodiments, the portions of the channel inserted into the body have channel has a maximal diameter D1 of at least 15 mm.

### Passageway Sleeve

According to some embodiments, the passageway sleeve generates and maintains an atraumatic opening between a target organ and the skin. According to some embodiments, the sleeve is shaped and made of a material that applies pressure/force on tissues engaging the sleeve, while preventing pressure point trauma. In some embodiments, the force is applied radially. In some embodiments, the sleeve applies an axial force in addition to a radial force. In some embodiments, applying the axial force is by retracting one or two ends of the sleeve.

According to some embodiments, the passageway sleeve is configured to have a distal portion secured to an internal surface of organ, while a proximal portion of the sleeve is disposed outside the organ. In some embodiments, the proximal portion is disposed outside the body, when the sleeve is secured to organ. In some embodiments, the proximal portion is pulled outside the body, after the sleeve is secured to organ.

According to some embodiments, the proximal portion is secured to the skin, prior to final fixation of the sleeve. According to some embodiments, securing the proximal portion to the skin prior to the distal portion enables pressure adjustment between the sleeve proximal and distal portion. In some embodiments, pre-fixating the proximal portion acts as a support when inserting the channel into the organ. In some embodiments, pre-fixating the proximal portion enables gradual releasing of the sleeve during insertion of the distal portion into the organ.

According to some embodiments, at least a portion of the sleeve is be made of a flexible polymer membrane such as thermoplastic polyester-polyurethane or silicon. In some embodiments, the sleeve has two semi-rigid polymer rings coupled to the wall of the sleeve. The length of the sleeve is adjustable by folding/unfolding the proximal ring toward/away of the inner distal ring. In some embodiments, folding of the proximal ring is by wrapping the sleeve around the outer proximal ring, until a desired length and/or pressure is achieved.

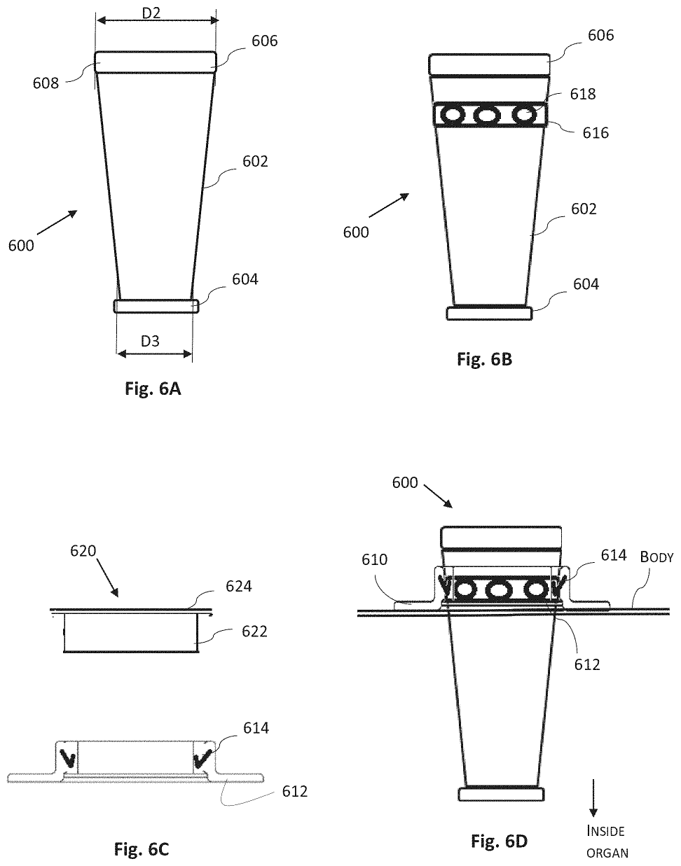

Turning to FIGS. 6A to 6B, which are simplified illustration of side views of an expanded passageway sleeve, according to some embodiments of the invention.

Passageway sleeve 600 has a main wall 602 and a distal fixation portion 604 configured to fixate inside an organ. In some embodiments, fixating portion 604 is encompassed within a distal portion of the wall 602 of sleeve 600.

According to some embodiments, distal fixating portion 604 is pliable. In some embodiments, fixating portion 604 includes a resilient ring coupled to the distal end of sleeve 600.

In some embodiments, fixating portion 604 includes a fully circular ring. In some embodiments, fixating portion 604 includes a semi-circular ring.

According to some embodiments, fixating portions 604 is shaped to be inserted inside a guiding channel (such as channels described elsewhere herein). In some embodiments, fixating portion 604 is disposed in a compressed/folded state inside the channel. In some embodiments, fixating portion 604 self-expands when released out of the channel.

According to some embodiments, distal fixating portion 604 is expandable. In some embodiments, fixating portion 604 includes an expandable shape memory member to expand fixating portion 604. In some embodiments, fixating portion 604 includes a circular spring to expand fixating portion 604. In some embodiments, fixating portion 604 is made of one or more inflatable portion. In some embodiments, fixating portion 604 is expandable by coupling fixating portion 604 to an expandable member shaped as an umbrella. In some embodiments, fixating portion 604 is expandable by coupling fixating portion 604 to an expandable balloon. In some embodiments, fixating portion 604 is made of a plurality of expandable portions.

According to some embodiments, sleeve 600 shaped to seal the organ to prevent leakage out of the organ. In some embodiments, sealing of the organ includes sealing an opening at the wall of the organ. In some embodiments, sleeve 600 seals the organ at a perforation rim created by inserting the channel into the organ. In some embodiments, sleeve 600 is configured seals the organ at the perforation rim after expanding fixating portion 604. In some embodiments, at least one of wall 602 and portion 604 of sleeve 600 apply an outward radial force on the tissue of the organ at the perforation rim. In some embodiments, sealing is by applying an axial force by at least one of wall 602 and portion 604 of sleeve 600 on the tissue of the organ at the perforation rim. In some embodiments, sealing is by applying a combination of the radial force and the axial force on the surfaces of the organ at the perforation rim. In some embodiments, at least a portion of sleeve 600 is axially elastic. In some embodiments, applying an axial force on surfaces of the organ is by an elastic retraction of sleeve 600 in an axial direction. In some embodiments, at least a portion of sleeve 600 is radially pliable. In some embodiments, applying an outward radial force on surfaces of the organ is by a pliable wall 602 stressed outwards by expanding sleeve 600 longitudinally. In some embodiments, fixation portion 604 includes a plurality of sealing portions for sealing the perforation rim.

According to some embodiments, as shown for example in FIGS. 6A-6B, wall 602 of sleeve 600 is formed into a conical shape. A potential advantage of a conical passageway is being shaped to have an outside opening larger than an inside opening, so that outside access is large enough for treatment (e.g. inserting of tools), while opening of the organ is reduced. A potential advantage of a conical passageway is being apply to increase or decrease of pressure applied at the tissues by axial movement of the passageway. A conical shape might make it easier to remove the passageway outside the organ, since pressure is reduced while pulling the conical sleeve outside the body.

According to some embodiments, sleeve 600 includes a proximal fixation portion 606 to secure sleeve 600 outside the body. In some embodiments, securing of sleeve 600 is by stretching proximal portion 606 outside the body, past the channel perforation. In some embodiments, stretching of sleeve 600 includes folding proximal portion 606 outside the body. In some embodiments, folding proximal portion 606 is by rolling wall 602 about proximal portion 606. In some embodiments, folding proximal portion 606 is by rolling wall 602 about the proximal edge of sleeve 600. In some embodiments, (as shown in FIGS. 6A-6B) proximal portion 606 include a pliable portion 608. In some embodiments, stretching wall 602 is after distal portion 604 is secured inside the organ, and is performed by rolling proximal portion 606 of sleeve 600 about pliable portion 608.

Turning to FIGS. 6B-6D, which show simplified side view illustrations of a passageway sleeve and a sleeve fastener, according to some embodiments of the invention.

According to some embodiments, the passageway system includes passageway sleeve 600 and a sleeve fastener 610. In some embodiments, securing proximal portion 606 of sleeve 600 outside the body is by coupling sleeve fastener 610 to sleeve 600. In some embodiments, stretching of sleeve 600 is by manipulating fastener 610 in respect to wall 602 of sleeve 600 and applying an axial force between proximal portion 606 and fixation portion 604. In some embodiments, stretching sleeve 600 is by manipulating fastener 610 after coupling fastener 610 to sleeve 600. In some embodiments, as shown for example in FIG. 6D, securing proximal portion 606 of sleeve 600 outside the body includes urging a bottom surface 612 of fastener 610 with the body.

According to some embodiments, as shown for example in FIGS. 6B-6D, sleeve fastener 610 includes a plurality of anchors 614 shaped to latch with a latching surface 616 disposed at the wall of sleeve 600. In some embodiments, anchors 614 are shaped to snap with latching surface 616. According to some embodiments, latching surface 616 is pre-formed formed at wall 602 of sleeve 600. In some embodiments, latching surface 616 is coupled to sleeve 600 during the process of securing sleeve 600 to the body. In some embodiments, latching surface includes a plurality of holes 618 formed to accept anchors 614.

According to some embodiments, the passageway system includes a securing cap 620 for shaped for fitting fastener 610. In some embodiments, fastener 610 is hollow and cap 620 is shaped to fit inside fastener 610. In some embodiments, cap 620 is shaped to fit over outside fastener 610.

In some embodiments, cap 620 is coupled to fastener 610 after sleeve 600 is secured to fastener 610. In some embodiments, fitting cap 620 is over proximal portion 604 that extend over fastener 610 after coupling fastener 610 and sleeve 600. In some embodiments, cap 620 has a hollow body 622 to maintain opening between proximal portion 606 and distal portion 604. In some embodiments, stoma bags are placed at cap 620 after securing sleeve 600 by using faster 610 can cap 200.

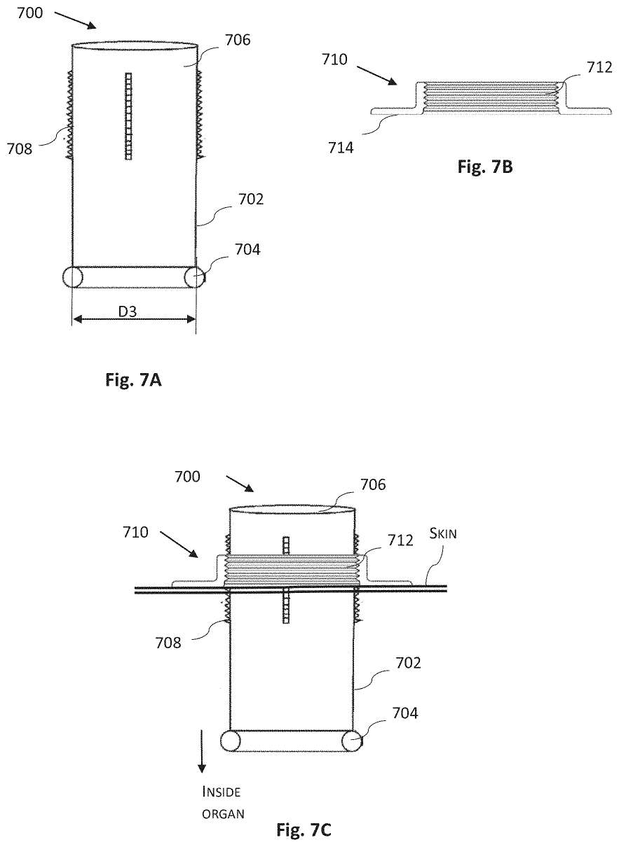

Turning to FIG. 7A to 7C, which are simplified illustration of side views of an expanded passageway sleeve, according to some embodiments of the invention.

According to some embodiments, as shown for example in FIG. 7A, the wall of passageway sleeve 700 is formed into a cylindrical shape. A potential advantage of a cylindrical passageway is a simpler design. In some embodiments, inserting tools in a straight direction is simpler when having a cylindrical passageway that which a conical passageway.

According to some embodiments, the maximal width D1 of the channel is less than the maximal width D3 of the distal opening of a secured passageway. In some embodiments, the maximal width D1 of the channel is less than the maximal diameter D3 of the opening of the fixation portion of secured passageway. In some embodiments, the maximal width of the perforation created by the channel is at least 5 to 10 mm less than the maximal diameter D3 of the opening of the fixation portion of secured passageway. In some embodiments, the maximal width D1 of the portions of the channel inserted inside the body is between 5 and 20 mm smaller than the maximal diameter D3 of a secured passageway. In some embodiments, the maximal width D1 of the portions of the channel inserted inside the body is between 10 and 15 mm smaller than the maximal diameter D3 of a secured passageway. In some embodiments, the maximal width D1 of the portions of the channel inserted inside the body is between 5 and 20 mm smaller than the maximal diameter D2 of a secured passageway at its distal end. In some embodiments, the maximal width D1 of the portions of the channel inserted inside the body is between 10 and 35 mm smaller than the maximal diameter of a secured passageway at its proximal end D2. In some embodiments, the proximal portion of the secured passageway has a diameter D2 of at least 10 mm. In some embodiments, the proximal portion of the secured passageway has a diameter D2 of at least 4 mm. In some embodiments, the proximal portion of the secured passageway has a diameter D2 of at least 15 mm. In some embodiments, the distal portion of the secured passageway has a diameter D3 of at least 10 mm. In some embodiments, the distal portion of the secured passageway has a diameter D3 of at least 4 mm. In some embodiments, the distal portion of the secured passageway has a diameter D3 of at least 15 mm.

FIGS. 7B-7C show simplified side view illustrations of a sleeve fastener, according to some embodiments of the invention.

As sleeve 600, sleeve 700 has a sleeve wall 702, a distal portion 704, and a proximal portion 706. According to some embodiments, one of the differences between sleeve 700 and sleeve 600 disclosed elsewhere herein is having a securing tread 708 formed at wall 702 of sleeve 700. In some embodiments, forming securing thread 708 on a cylindrical wall 702 as in passageway sleeve 700 is simpler that forming such thread on a conical wall.

One of the difference between sleeve fastener 710 and sleeve fastener 610 is coupling and manipulating of sleeve fastener 710 in respect to sleeve 700 by using a screw mechanism. A potential advantage of securing sleeve 700 by screwing a fattener 710 is applying a controlled pressure by fastener 710 on the skin. Screwing fastener 710 potentially reduces the risk of accidental releasing of the proximal portion 706 of sleeve 700.

According to some embodiments, sleeve fastener 710 has an internal thread 712 and stretching of sleeve 700 is by screwing sleeve fastener 710 about a longitudinal axis of sleeve 700 using threads 708 and 712. In some embodiments, fastener 710 is used for securing sleeve 700 to the skin by engaging bottom surface 714 of fastener 710 with the skin.

According to some embodiments, the passageway sleeve (e.g. 600 or 700) is lubricated prior to insertion into the organ. In some embodiments, lubrication of the sleeve is by providing a lubricated material via the channel after inserting the sleeve within the channel. According to some embodiments, lubricating material includes medication. In some embodiments, lubricating material includes sealing material.

According to some embodiments, the passageway insertion system includes a sleeve magazine (not shown) shaped to load the sleeve into the channel. In some embodiments, the magazine is shaped and size be inserted into the channel. In some embodiments, the magazine is shaped to receive the sleeve and deliver the sleeve in a folded shape inside the channel. In some embodiments, the load of the sleeve via the magazine is done prior to the insertion of the channel. In some embodiments, the load of the sleeve via the magazine is done after the insertion of the channel to the body, once channel is located in the target organ.

### Pre Fixating Organ To Body

According to some embodiments, the procedure of inserting a passageway sleeve into an abdomen organ includes pre-fixating the organ to the abdomen wall.

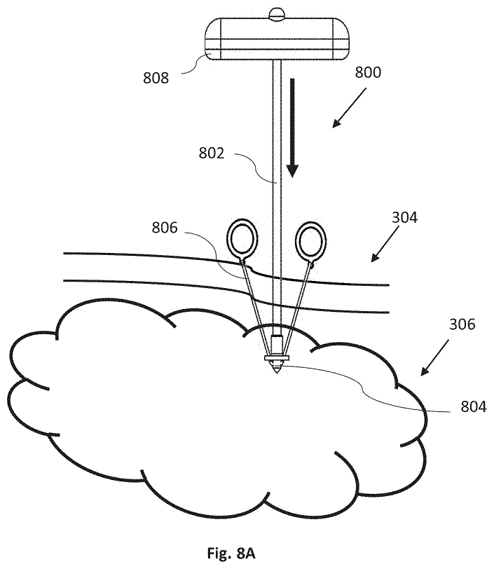

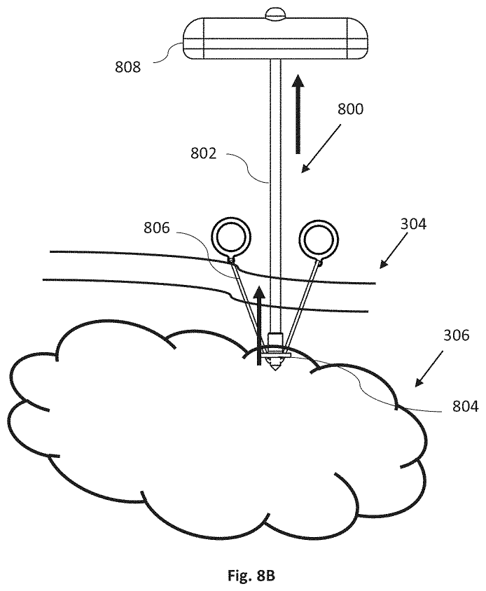

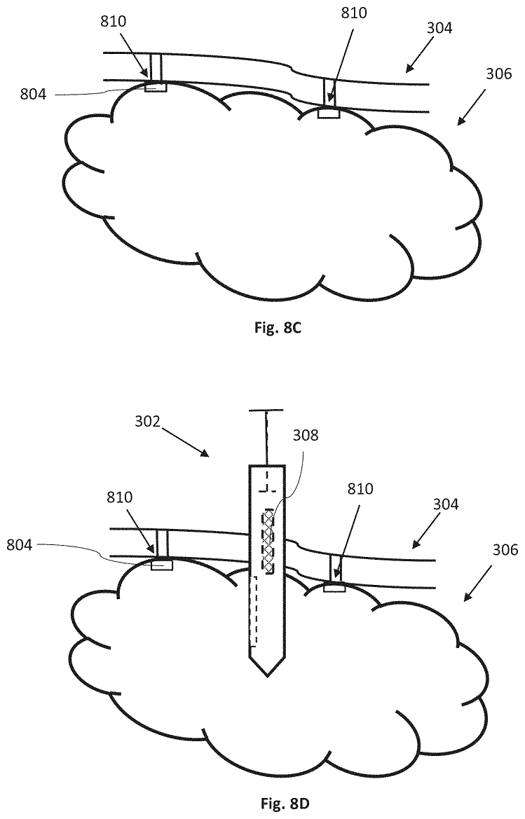

Turning to FIGS. 8A-8D which show side view simplified illustrations of an organ pre-fixation system used to pre-fixate an abdomen organ to the abdomen wall, according to some embodiments of the invention.

According to some embodiments, as shown for example in FIG. 8A, organ fixation system 800 includes a detachable organ securing head 804 coupled to mandrel body 802.

According to some embodiments, mandrel 802 has a sharp distal tip for penetrating the wall of organ 306 by force applied by an operator. In some embodiments, head 804 is disposed at the tip of mandrel 802 and has a sharp tip for penetrating the wall of organ 306.

According to some embodiments, mandrel 802 includes one or more lumens to enable bowel air/gas/fluid release to decrease intra bowel pressure and avoid bowel rupture in case required.

According to some embodiments, head 804 is pre-connected with stitching wires 806 to be used to fixate and stich head 804 to the skin 304.

According to some embodiments, the procedure for fixating organ 306 includes marking the skin with an identified spot for inserting mandrel 802 with the sharped detachable head 804. Optionally, inserting the head 804 is preceded by cleaning the skin with antiseptic and local anesthesia is administered. Optionally, the patient is sedated. In some embodiments, inserting of head 804 is under imaging means.

According to some embodiments, as shown for example in FIG. 8B, when head 804 is located in the bowel, the physician releases mandrel 802 distally outside the body 304. In some embodiments, releasing mandrel 802 detaches head 804 out of the organ fixation system 800, leaving the detach head 804 in organ 306. In some embodiments, head 804 is detached by a button located on the mandrel head 808.

Optionally the physician is repeating the organ pre-fixation procedure. In some embodiments, fixation is at 3 to 6 spots.

Optionally, the organ is pre-fixated in a distance of 1-3 cm from the inner abdominal wall. In some embodiments, pre-fixation attaches some portions of the organ to the inner abdominal wall, while other portions of the organ are attached without engaging the inner abdominal wall. Optionally, once all heads 604 are fixed to the skin 806, the physician marks the passageway insertion spot and performs an incision as preparation for the insertion of the trocar.

FIG. 8D is an example embodiment, showing an insertion of channel 302 into organ 306 after organ 306 is secured to the abdomen and organ fixation system 800 is removed (as shown in FIG. 8C). According to some embodiments, an organ securing system 800 is used to couple organ 306 to inner abdomen wall 304 at one or more securing points 810, prior to inserting channel 302. In some embodiments, organ 306 is secured to the body at 3 securing points 810. In some embodiments, channel 302 is inserted into the abdomen through an incision made between securing points 810.

### General

It is expected that during the life of a patent maturing from this application many relevant passageway sleeves will be developed; the scope of the term passageway sleeve is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”. The term “consisting essentially of” means than the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise.

Throughout this application, embodiments of this invention may be presented with reference to a range format. It should be understood than the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority documents of this application are hereby incorporated herein by reference in their entirety.

1. A method of inserting a passageway sleeve into an internal organ, comprising:
inserting a channel into an organ by perforating the skin and the wall of the organ;
releasing the passageway sleeve inside the organ;
fixating a fixation portion of the passageway sleeve to the wall of the organ;
removing the channel outside the body; and
expanding the passageway sleeve to produce a passageway between outside the body and inside the organ.

2. The method of claim 1, comprising:
advancing a tool into the organ from a proximal portion of the passageway sleeve.

3. A method of inserting a flexible sleeve as a passageway into an internal organ, comprising:
inserting a channel into an organ by perforating the wall of the organ;
releasing a distal fixation portion of the passageway sleeve inside the organ;
fixating the distal fixation portion to the wall of the organ; and
pulling a proximal portion of the passageway sleeve outside the body to stretch the sleeve, wherein the stretched sleeve:
a) generates a passageway between the proximal end of the sleeve at the outer body portion and the distal portion of the sleeve inside the organ, and
b) seals the organ at a perforation rim created by said perforating.

4. The method of claim 3, comprising:
advancing the fixation portion inside the channel to a release port formed at a wall of the channel; and releasing the fixation portion out of the release port.

5. The method according to claim 3, comprising:
securing the proximal portion of the sleeve to the body.

6. The method according to claim 3, comprising:
removing content out of the internal organ via the passageway.

7. The method according to claim 3, comprising:
removing the channel outside the organ and the body prior to the pulling of the proximal portion.

8. The method according to claim 3, comprising:
pre-fixating the organ to the body at one or more locations, prior to inserting the channel.

9. The method of claim 8, comprising making an incision between the one or more locations.

10. The method according to claim 3, comprising:
reducing the size of the fixation portion of the passageway sleeve; and
removing the passageway sleeve outside the organ.

11. The method according to claim 3, wherein said inserting is without anesthesia.

12. (canceled)

13. An intra-body passageway system, comprising:
a channel, comprising:
an elongated barrel having a sleeve release port, and
a tip at a distal end of the channel, sized and shaped to penetrate an organ; and
a passageway sleeve, inserted at least partially within the barrel, comprising:
a flexible wall, between a distal end and a proximal end of the sleeve, and
a resilient fixation portion, integrated to the distal end of the sleeve; and
wherein the sleeve release port is shaped and sized to release the fixation portion out of the channel.

14. A system of claim 13, wherein and wherein the fixation portion is expandable out of the channel, via the sleeve release port.

15. A system according to claim 13, wherein the portion of the barrel configured to penetrate the organ has a maximal width of at least 10 mm.

16. A system according to claim 13, wherein the channel comprises a plunger, and the sleeve is slideable by the plunger.

17. A system according to claim 13, wherein the wall of the passageway sleeve has a conical shape between the fixation portion and the proximal portion of the passageway sleeve, when unfolded.

18. A system according to claim 13, comprising a sleeve fastener shaped to couple to a proximal portion of the passageway sleeve to secure the sleeve to the body.

19. (canceled)

20. A system according to claim 13, wherein the barrel comprises a sleeve orientation flange, protruding from the sleeve release port to control the orientation of the fixation portion, by interrupting the expansion of the fixation portion out of the sleeve release port.

21. A system according to claim 13, wherein the tip is pivotally coupled to the barrel and/or wherein the tip is detachable of the barrel. 22-24. (canceled)

25. A system according to claim 13, wherein the fixation portion is pliable and self-expands when not confined, and/or wherein the fixation portion includes one or more resilient rings coupled to the distal end of sleeve. 26-30. (canceled)
